(19) United States
(12) Patent Application Publication
(10) Pub. No.: US 2026/0175029 A1
(43) Pub. Date: Jun. 25, 2026

(54) DEEP BRAIN STIMULATION ALGORITHM SYSTEM, OPERATION METHOD OF THE SAME AND NON-TRANSITORY COMPUTER READABLE STORAGE MEDIUM

(71) Applicant: INDUSTRIAL TECHNOLOGY RESEARCH INSTITUTE, HSINCHU (TW)

(72) Inventors: Chang-Lin HU, Kaohsiung City (TW); Pei-Ling KANG, Hsinchu County (TW); Kuan-Yi LU, Tainan City (TW); Hung-Wei LIN, Chiayi County (TW); Chun-Hung LIEN, Taipei City (TW)

(21) Appl. No.: 19/001,386

(22) Filed: Dec. 24, 2024

(30) Foreign Application Priority Data
Dec. 23, 2024 (TW) 113150280

(51) Int. Cl. A61N 1/36; A61N 1/372; G16H 20/40

(52) U.S. Cl. CPC A61N 1/36139; A61N 1/37217; G16H 20/40

(57) ABSTRACT

A deep brain stimulation algorithm system is provided. The deep brain stimulation algorithm system comprises a deep brain stimulation device configured to generate an electrical stimulation signal to a subject and adjust the electrical stimulation signal based on a stimulation parameter. The deep brain stimulation device comprises a signal sensing module, a control module, a personalization module and an adjustment module. The signal sensing module is configured to sense a local field potential signal from the subject. The control module is configured to generate the stimulation parameter and adjust the stimulation parameter based on an adjustment parameter. The personalization module is configured to generate a prediction result based on the medical data and the local field potential signal. The adjustment module is configured to compare the local field potential signal and the prediction result by using a full spectrum analysis, so as to generate the adjustment parameter.

### BACKGROUND

### Technical Field

The present disclosure relates to a deep brain stimulation algorithm system, operation method of the same and a non-transitory computer readable storage medium.

### Description of Related Art

As the average life span of modern humans increases, the number of people suffering from chronic neurodegenerative diseases (e.g., Alzheimer's disease, Parkinson's disease, etc.) due to the degeneration of the central nervous system is also gradually increasing. Therefore, deep brain stimulation (DBS) is widely used in the treatment of central neuropathy.

However, the brain waves sensed by currently widely used deep brain electrical stimulation systems are usually limited to beta signals of a local field potential (LFP) signal of subthalamus nucleus (STN), while the symptoms caused by other signals of the LFP signal are ignored, resulting in less effective treatment than expected. In addition, the features of a subject's LFP signal may vary from person to person, but currently used electrical stimulation systems lack a mechanism to adjust for this individual difference, resulting in poor adaptability. Therefore, how to improve the response capabilities of deep brain electrical stimulation systems is one of the topics in this field.

### SUMMARY

A deep brain stimulation algorithm system is provided in the present disclosure. The deep brain stimulation algorithm system comprises a deep brain stimulation device. The deep brain stimulation device is configured to generate an electrical stimulation signal to a subject and adjust the electrical stimulation signal based on a stimulation parameter. The deep brain stimulation device comprises a signal sensing module, a control module, a personalization module and an adjustment module. The signal sensing module is configured to sense a local field potential signal from the subject after the subject receives the electrical stimulation signal. The control module is coupled to the signal sensing module, configured to generate the stimulation parameter, and configured to adjust the stimulation parameter based on an adjustment parameter. The personalization module is coupled to the signal sensing module, and configured to generate a prediction result based on a medical data and the local field potential signal. The adjustment module is coupled to the signal sensing module, the control module and the personalization module, and configured to compare the local field potential signal and the prediction result by using a full spectrum analysis, so as to generate the adjustment parameter to the control module.

An operation method of a deep brain stimulation algorithm system is provided in the present disclosure. The operation method comprises: generating, by a deep brain stimulation device, an electrical stimulation signal to a subject; sensing, by a signal sensing module of the deep brain stimulation device, a local field potential signal from the subject; generating, by a control module of the deep brain stimulation device, a stimulation parameter; generating, by a personalization module of the deep brain stimulation device, a prediction result based on a medical data and the local field potential signal; comparing, by an adjustment module of the deep brain stimulation device, the local field potential signal and the prediction result by using a full spectrum analysis, so as to generate an adjustment parameter to the control module; adjusting, by the control module, the stimulation parameter based on the adjustment parameter; and adjusting, by the deep brain stimulation device, the electrical stimulation signal based on the stimulation parameter.

A non-transient computer readable storage medium is provided in the present disclosure. The non-transient computer readable storage medium stores a plurality of computer readable instructions. when the plurality of computer readable instructions are executed for performing a deep brain stimulation algorithm on a subject by one or a plurality of processors, the one or the plurality of processors is configured to perform the following operations: generating an electrical stimulation signal to the subject; sensing a local field potential signal from the subject; generating a stimulation parameter; generating a prediction result based on a medical data and the local field potential signal; comparing the local field potential signal and the prediction result by using a full spectrum analysis, so as to generate an adjustment parameter; adjusting the stimulation parameter based on the adjustment parameter; and adjusting the electrical stimulation signal based on the stimulation parameter.

### BRIEF DESCRIPTION OF THE DRAWINGS

The present disclosure can be more fully understood by reading the following detailed description of the embodiment, with reference made to the accompanying drawings as follows.

FIG. 1 is a functional block diagram of a deep brain stimulation algorithm system in accordance with some embodiments of the present disclosure.

FIG. 2 is a flowchart of an operation method of a deep brain stimulation algorithm system in accordance with some embodiments of the present disclosure.

FIG. 3 is a flowchart of some steps of the operation method of the deep brain stimulation algorithm system in accordance with some embodiments of the present disclosure.

FIG. 4 is a flowchart of some steps of the operation method of the deep brain stimulation algorithm system in accordance with some embodiments of the present disclosure.

FIG. 5A is a schematic diagram of a local field potential (LFP) signal in accordance with some embodiments of the present disclosure.

FIG. 5B is a schematic diagram of a signal converted from a portion of the LFP signal of FIG. 5A in accordance with some embodiments of the present disclosure.

FIG. 5C is a schematic diagram of a signal converted from a portion of the LFP signal of FIG. 5A in accordance with some embodiments of the present disclosure.

### DETAILED DESCRIPTION

For purposes of explanation, numerous specific details are set forth below to enable a thorough understanding of the embodiments of the present disclosure. It will be apparent, however, that one or more embodiments may be practiced without these specific details. In other cases, well-known structures and devices are only shown schematically in order to simplify the drawings. Reference will now be made in detail to the present embodiments of the disclosure, examples of which are illustrated in the accompanying drawings.

In the present disclosure, when an element is referred to as “connected”, it may mean “electrically connected” or “signal connected”. When an element is referred to as “coupled”, it may mean “electrically coupled” or “signal coupled”. “Connected” or “coupled” can also be used to indicate that two or more components operate or interact with each other. As used in the present disclosure, the singular forms “a”, “one” and “the” are also intended to include plural forms, unless the context clearly indicates otherwise. It will be further understood that when used in this specification, the terms “comprises (comprising)” and/or “includes (including)” designate the existence of stated features, steps, operations, elements and/or components, but the existence or addition of one or more other features, steps, operations, elements, components, and/or groups thereof are not excluded.

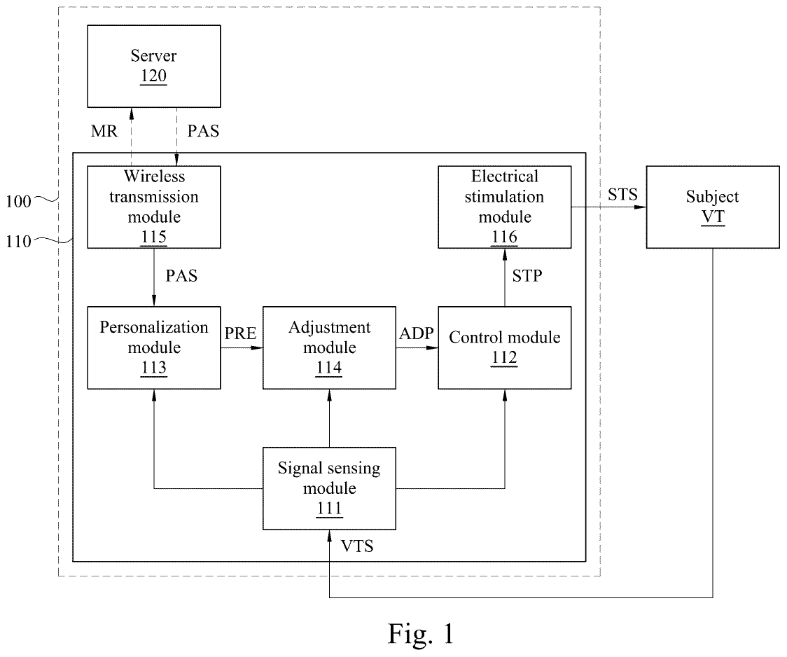

FIG. 1 is a functional block diagram of a deep brain stimulation algorithm system 100 in accordance with some embodiments of the present disclosure. In some embodiments, the deep brain stimulation algorithm system 100 comprises a deep brain stimulation device 110 and a server 120.

Specifically, the term “deep brain stimulation” used in the present disclosure is an operation performed by implanting electrode leads into deep layers (e.g., subthalamus nucleus (STN) or globus pallidus internal segment (GPi)) of a brain and applying electricity to them, wherein the term “deep brain” refers to the aforementioned STN or Gpi.

The deep brain stimulation device 110 is connected to a subject VT, and is configured to transmit an electrical stimulation signal STS to the subject VT and receive a local field potential (LFP) signal VTS from the subject VT. In some embodiments, the deep brain stimulation device 110 is connected to the server 120 by signals (i.e., signals are transmitted through wireless communication, marked with dotted lines in the figure) to exchange data with server 120. In some embodiments, the deep brain stimulation device 110 comprises a signal sensing module 111, a control module 112, a personalization module 113 and an adjustment module 114.

The signal sensing module 111 is coupled to the control module 112, the personalization module 113 and the adjustment module 114, and is configured to sense the local field potential signal VTS from the subject VT after the subject VT receives the electrical stimulation signal STS.

In some embodiments, the local field potential signal VTS comprises various bands of brain wave signals in the subthalamic nucleus (STN), such as alpha brain wave signal (with frequency of about 8-13 Hz), beta brain wave signal (with frequency of about 13-35 Hz), gamma brain wave signal (with frequency of about 35-90 Hz), etc. Abnormal increases in the power of these brain wave signals may cause different symptoms. For example, an increase in the power of alpha brain wave signals may cause symptoms of tremor, an increase in the power of beta brain wave signals may cause symptoms of bradykinesia, and an increase in the power of gamma brain wave signals may cause symptoms of dyskinesia. In order to alleviate these symptoms, the deep brain stimulation device 110 provides corresponding electrical stimulation signals STS to the subject VT.

The control module 112 is coupled to the signal sensing module 111 and the adjustment module 114, and is configured to generate a stimulation parameter STP. In some embodiments, the control module 112 generates the stimulation parameter STP based on a medical data MR (e.g., the age, gender, race, living environment, images of computed tomography, images of magnetic resonance imaging, etc. of the subject VT) and/or the local field potential signal VTS received from the signal sensing module 111, wherein the stimulation parameter STP is related to the intensity of the electrical stimulation signal STS output by the deep brain stimulation device 110. However, it should be noted that the method of generating the stimulation parameter STP disclosed in the present disclosure (i.e., generating the stimulation parameter STP based on the medical data MR and/or the local field potential signal VTS) is only an example, and is not intended limit the present disclosure. Other methods of generating the stimulation parameter STP based on any individual physiological information are within the scope of the present disclosure. By adjusting the stimulation parameters STP based on the medical data MR of the subject VT, the deep brain stimulation device 110 of the present disclosure can achieve the characteristic of “personalization”.

In some embodiments, the control module 112 comprises an artificial intelligence (AI) model. This artificial intelligence model is configured to select a specific algorithm from a plurality of algorithms (e.g., switch control algorithm, proportional control algorithm, or dual threshold control algorithm), and the control module 112 may use the selected algorithm to generate the stimulation parameter STP. For example, in an embodiment where the stimulation parameter STP needs to be adjusted between a plurality of thresholds, the artificial intelligence model will select the dual threshold control algorithm (or a multi-threshold control algorithm) as the specific algorithm to generate the stimulation parameter STP.

The personalization module 113 is coupled to the signal sensing module 111 and the adjustment module 114, and is configured to generate a prediction result PRE to the adjustment module 114 based on the medical data MR and the local field potential signal VTS received from the signal sensing module 111. The prediction result PRE is used to indicate the ideal spectrum of the local field potential signal VTS when the subject VT receives the current deep brain electrical stimulation.

In some embodiments, the personalization module 113 is further configured to receive a personalization adjustment signal PAS from the server 120 and adjust the prediction result PRE based on the personalization adjustment signal PAS. Regarding the detailed operation of the personalization module 113 using the personalization adjustment signal PAS to adjust the prediction result PRE, please refer to the description in the subsequent paragraphs.

The adjustment module 114 is coupled to the signal sensing module 111, the control module 112 and the personalization module 113, and is configured to compare the local field potential signal VTS and the prediction result PRE by using a full spectrum analysis, so as to generate an adjustment parameter ADP to the control module 112. In some embodiments, the full spectrum analysis is used to analyze the difference between the wave patterns in the spectrum of the local field potential signal VTS and the prediction result PRE (e.g., a difference of 5%), wherein the difference is positively related to the adjustment parameter. In other words, the greater the difference between the local field potential signal VTS and the prediction result PRE, the greater the adjustment parameter ADP will be.

In some embodiments, after the control module 112 receives the adjustment parameter ADP from the adjustment module 114, the control module 112 will adjust the stimulation parameter STP based on the adjustment parameter ADP, thereby changing the intensity of the electrical stimulation signal STS output by the deep brain stimulation device 110, wherein the change of the electrical stimulation signal STS is positively related to the adjustment parameter ADP. In some embodiments, the adjustment module 114 uses a fuzzy decision method to generate the adjustment parameter ADP based on the error between the local field potential signal VTS and the prediction result PRE.

By generating the adjustment parameter ADP based on the local field potential signal VTS and the prediction result PRE to adjust the stimulation parameter STP, the deep brain stimulation device 110 of the present disclosure can achieve the characteristic of “self-adaptive”. In addition, in some embodiments, after finishing the adjustment of the stimulation parameter STP and outputting the adjusted electrical stimulation signal STS, the deep brain stimulation device 110 will sense the local field potential signal VTS of the subject VT again and repeat the aforementioned prediction and comparison operations, and thus the deep brain stimulation device 110 of the present disclosure can achieve the characteristic of “closed-loop”.

By using full spectrum analysis to compare the local field potential signal VTS and the prediction result PRE, multiple most obvious features (e.g., power, mean, standard deviation, skewness, kurtosis, entropy, etc.) in all frequency bands (e.g., 0 -200 Hz) related to the local field potential signal of the subthalamic nucleus can be extracted, and it can be determined whether these features exist in the local field potential signal VTS of the subject VT, thereby improving the accuracy of symptom judgment.

In some embodiments, the adjustment module 114 is configured to generate the adjustment parameter ADP to the control module 112 in each adjustment cycle (e.g., every minute). In other embodiments, the adjustment module 114 is configured to generate the adjustment parameter ADP to the control module 112 when receiving an adjustment command (e.g., through manual input). Through the above operations of instructing the adjustment module 114 to generate the adjustment parameter ADP, the deep brain stimulation device 110 of the present disclosure can achieve the characteristic of “semi/fully automatic”.

In some embodiments, the deep brain stimulation device 110 further comprises a wireless transmission module 115. The wireless transmission module 115 is coupled between the personalization module 113 and the server 120, and is configured to provide a signal path between the deep brain stimulation device 110 and the server 120, so that the deep brain stimulation device 110 and the server 120 can transmit data to each other through wireless communication.

In some embodiments, the deep brain stimulation device 110 further comprises an electrical stimulation module 116. The electrical stimulation module 116 is coupled between the control module 112 and the subject VT, and is configured to generate the electrical stimulation signal STS to the subject VT based on the stimulation parameter STP. As mentioned above, after the control module 112 receives the adjustment parameter ADP and adjusts the stimulation parameter STP, the electrical stimulation module 116 will correspondingly adjust the intensity of the electrical stimulation signal STS based on the adjustment of the stimulation parameter STP. In some embodiments, the electrical stimulation module 116 comprises components such as batteries, electrodes implanted deep in the brain, circuits and wires for electrical stimulation.

In some embodiments, each of the signal sensing module 111, the control module 112, the personalization module 113, the adjustment module 114, the wireless transmission module 115 and the electrical stimulation module 116 can be implemented with electronic components, circuits, electronic devices or any combination of the above.

The server 120 is coupled to the deep brain stimulation device 110 (and the wireless transmission module 115 therein). The server 120 stores disease statistics, wherein the disease statistics record statistical data on the various characteristics of patients (e.g., age, gender, race, living environment, etc.) of different diseases. In some embodiments, the server 120 is configured to receive the medical data MR from the deep brain stimulation device 110 through the wireless transmission module 115, and generate the personalization adjustment signal PAS to the personalization module 113 based on the medical data MR and the disease statistics, so that the personalization module 113 can adjust the prediction result PRE based on the personalization adjustment signal PAS. By comparing the medical data MR and the disease statistics, the personalization module 113 can generate more objective prediction result PRE.

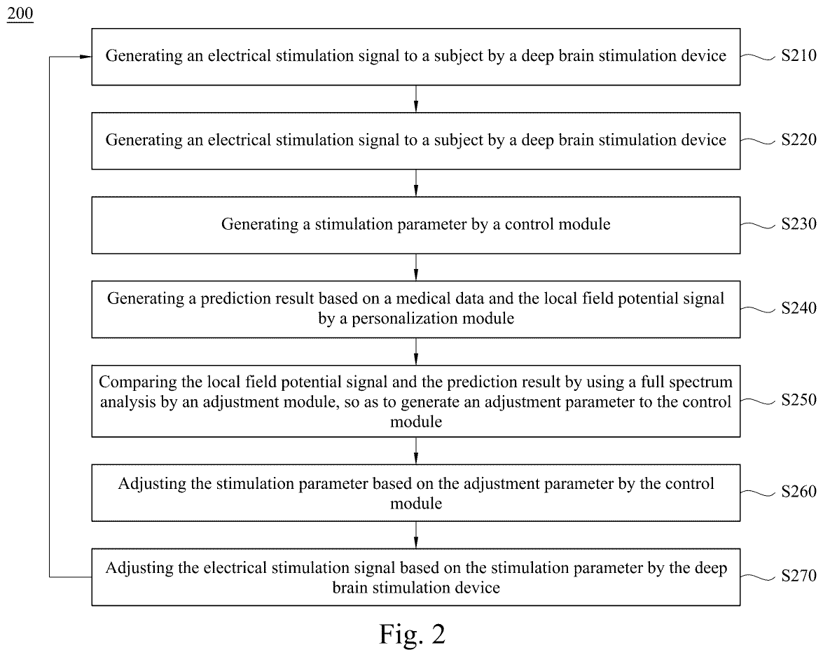

FIG. 2 is a flowchart of an operation method 200 of a deep brain stimulation algorithm system in accordance with some embodiments of the present disclosure. In some embodiments, the operation method 200 is suitable for a deep brain stimulation algorithm system (such as the deep brain stimulation algorithm system 100 of the present disclosure) and comprises steps S210, S220, S230, S240, S250, S260 and S270.

In step S210, an electrical stimulation signal is generated to a subject based on a stimulation parameter by an electrical stimulation module (e.g., the electrical stimulation module 116 of FIG. 1) of a deep brain stimulation device (e.g., the deep brain stimulation device 110 of FIG. 1). Next, step S220 will be performed.

In step S220, a local field potential signal from the subject is sensed by a signal sensing module (e.g., the signal sensing module 111 of FIG. 1) of the deep brain stimulation device. Next, step S230 will be performed.

In step S230, a stimulation parameter is generated by a control module (e.g., the control module 112 of FIG. 1) of the deep brain stimulation device (e.g., based on a medical data and/or the local field potential signal, but the present disclosure is not limited thereto). Next, step S240 will be performed.

In step S240, a prediction result is generated by a personalization module (e.g., the personalization module 113 of FIG. 1) of the deep brain stimulation device based on a medical data and the local field potential signal. Next, step S250 will be performed.

In step S250, the local field potential signal and the prediction result are compared by using a full spectrum analysis by an adjustment module (e.g., the adjustment module 114 of FIG. 1) of the deep brain stimulation device, so that the adjustment module can generate an adjustment parameter to the control module. Next, step S260 will be performed.

In step S260, the stimulation parameter is adjusted by the control module based on the adjustment parameter. Next, step S270 will be performed. In step S270, the electrical stimulation signal is adjusted by the deep brain stimulation device based on the stimulation parameter. After step S270, step S210 will be performed again, so as to transmit the adjusted electrical stimulation signal to the subject.

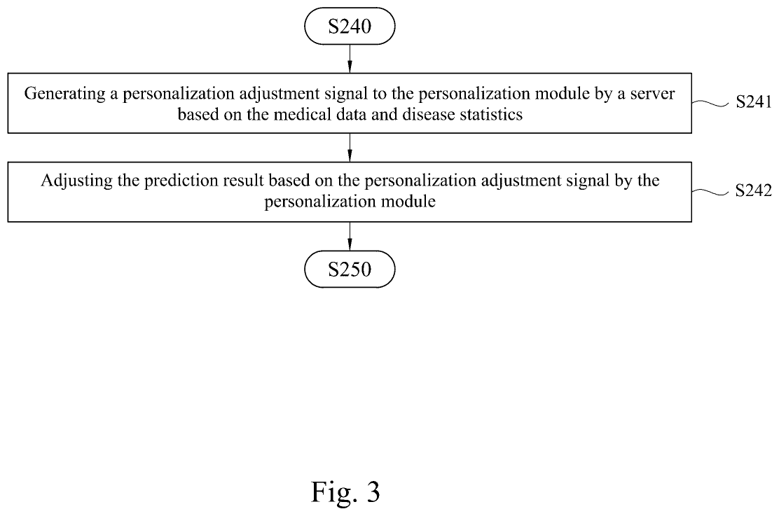

In some embodiments, there may be more steps between the steps S240 and S250 of FIG. 2. Please refer to FIG. 3. FIG. 3 is a flowchart of the steps between steps S240 and S250 of the operation method 200 of the deep brain stimulation algorithm system in accordance with some embodiments of the present disclosure. In some embodiments, there are steps S241 and S242 between steps S240 and S250.

In step S241, a personalization adjustment signal is generated to the personalization module by a server (e.g., the server 120 of FIG. 1) based on the medical data and disease statistics. Next, step S242 will be performed.

In step S242, the prediction result is adjusted by the personalization module based on the personalization adjustment signal. Next, step S250 will be performed.

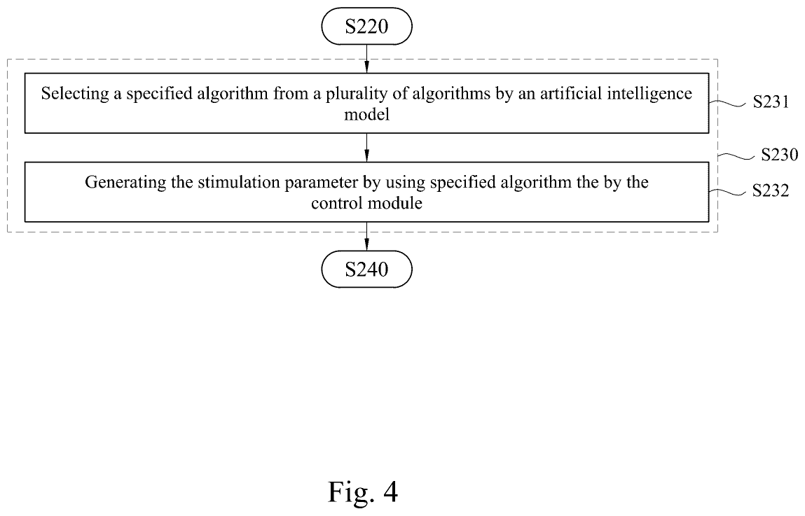

In some embodiments, the step S230 of FIG. 2 further comprises some detailed steps. Please refer to FIG. 4. FIG. 4 is a flowchart of the steps within the step S230 of the operation method 200 of the deep brain stimulation algorithm system in accordance with some embodiments of the present disclosure. In some embodiments, step S230 comprises steps S231 and S232.

In step S231, a specified algorithm (also called a selected algorithm) is selected from a plurality of algorithms by an artificial intelligence model. Next, step S232 will be performed.

In step S232, the stimulation parameter is generated by using the specified algorithm by the control module (e.g., based on the medical data and/or the local field potential signal, but the present disclosure is not limited thereto). Next, step S240 will be performed.

It should be noted that the number and order of steps in the operation method 200 of the present disclosure are only examples, and are not intended to limit the present disclosure. Other numbers and orders of steps are within the scope of the present disclosure. In some embodiments, steps S230 and S240 can be performed synchronously. In other embodiments, step S230 may be performed after step S240.

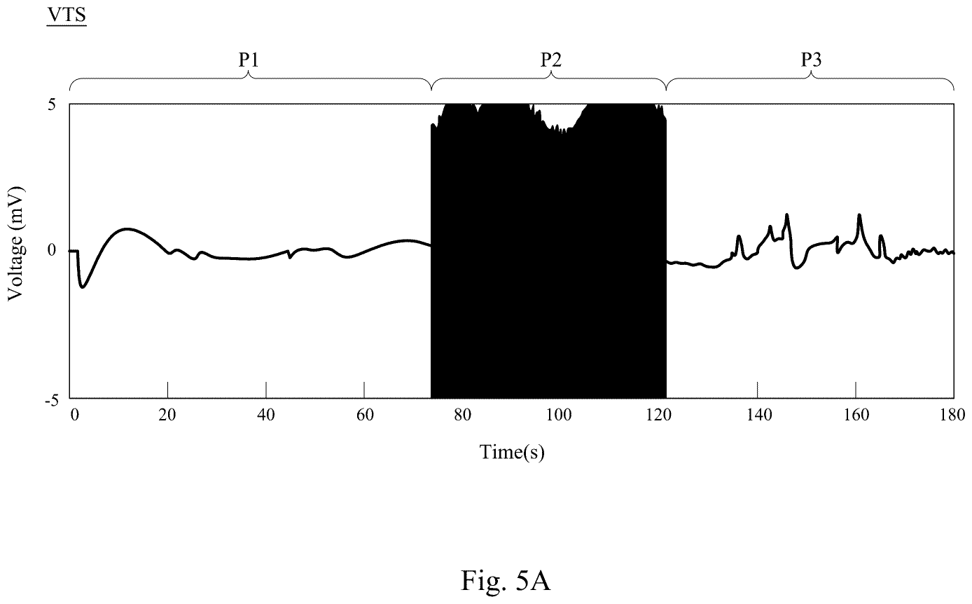

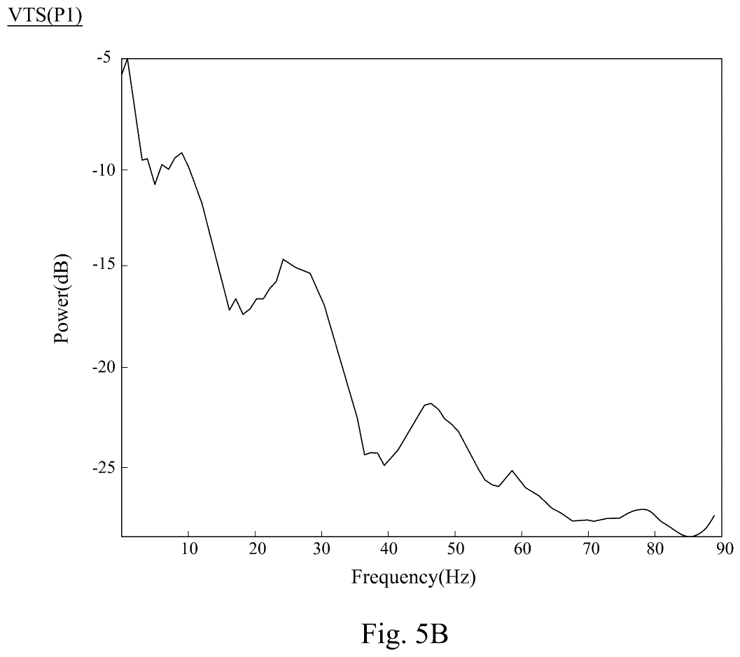

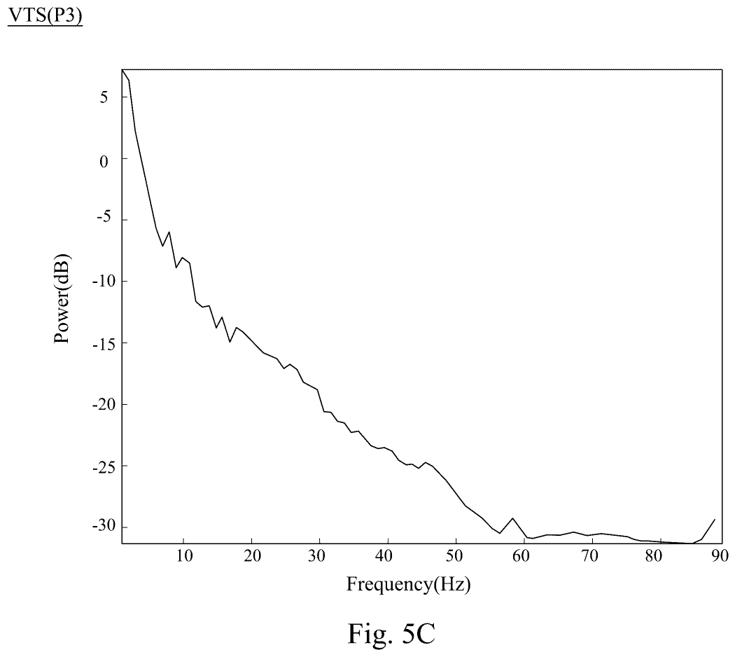

FIG. 5A is a schematic diagram in the time domain of the local field potential signal VTS sensed by the deep brain stimulation algorithm system 100 in accordance with some embodiments of the present disclosure. In some embodiments, periods P1, P2 and P3 respectively represent the periods before the subject VT receives deep brain electrical stimulation, during the period of receiving deep brain electrical stimulation, and after receiving deep brain electrical stimulation.

In some embodiments, the deep brain stimulation algorithm system 100 can use Fourier transform to convert the time domain signal of FIG. 5A into a frequency domain signal, so as to help determining the type of abnormal brain waves. In some embodiments, the aforementioned Fourier transform can be performed by the signal sensing module 111 of the deep brain stimulation algorithm system 100.

After converting the time domain schematic diagram into a frequency domain schematic diagram through Fourier transform, the brain wave signal band where symptoms may occur can be judged by observing the wave peaks in the schematic diagram, thereby improving the accuracy and efficiency of judgment. Please refer to FIG. 5B. FIG. 5B is a schematic diagram of a signal converted from the period P1 of the local field potential signal VTS of FIG. 5A through Fourier transform in accordance with some embodiments of the present disclosure. As shown in FIG. 5B, after converting the time domain schematic diagram of FIG. 5A into the frequency domain schematic diagram of FIG. 5B, it can be clearly observed that wave peaks appear at the frequencies of 11, 26 and 48 Hz. Therefore, the symptoms that the subject experienced before receiving deep brain electrical stimulation can be determined according to these frequencies.

Next, please refer to FIG. 5C. FIG. 5C is a schematic diagram of a signal converted from the period P3 of the local field potential signal VTS of FIG. 5A through Fourier transform in accordance with some embodiments of the present disclosure. As shown in FIG. 5C, the peaks sensed in the wave bands where the peaks appear in FIG. 5B (i.e., frequencies of 11, 26, and 48 Hz) have reduced/disappeared. Therefore, it can be judged that the original symptoms of the subject have been relieved after receiving deep brain electrical stimulation.

The present disclosure provides a non-transient computer readable storage medium storing a plurality of computer readable instructions, when the plurality of computer readable instructions are executed by one or a plurality of processors, the one or the plurality of processors is configured to perform the operation method 200 described above. In some embodiments, the non-transient computer readable storage medium is an electronic, magnetic, optical, electromagnetic, infrared, and/or a semiconductor system (or apparatus or device). For example, the computer readable storage medium comprises a semiconductor or solid-state memory, a magnetic tape, a removable computer diskette, a random access memory (RAM), a read-only memory (ROM), a rigid magnetic disk, and/or an optical disk. In some embodiments using optical disks, the computer readable storage medium comprises a compact disk-read only memory (CD-ROM), a compact disk-read/write (CD-R/W) and/or a digital video disc (DVD).

Through the deep brain stimulation algorithm system 100, its operation method 200 and the non-transitory computer readable storage medium of the present disclosure, the sensing range and accuracy of deep brain electrical stimulation can be improved through full-spectrum analysis, and the electrical stimulation signal can be adjusted according to various characteristics of subjects, thereby realizing the optimization of a deep brain stimulation algorithm with the characteristics of self-adaptive, personalization and closed-loop.

It will be apparent to those skilled in the art that various modifications and variations can be made to the disclosed embodiments. It is intended that the specification and examples be considered as exemplars only, with a true scope of the disclosure being indicated by the following claims and their equivalents.

What is claimed is:

1. A deep brain stimulation algorithm system, comprising:
a deep brain stimulation device, configured to generate an electrical stimulation signal to a subject, wherein the deep brain stimulation device comprises:
a signal sensing module, configured to sense a local field potential signal from the subject after the subject receives the electrical stimulation signal;
a control module, configured to generate a stimulation parameter;
a personalization module, configured to generate a prediction result based on a medical data and the local field potential signal; and
an adjustment module, configured to compare the local field potential signal and the prediction result by using a full spectrum analysis, so as to generate an adjustment parameter to the control module,
wherein the control module is further configured to adjust the stimulation parameter based on the adjustment parameter, and the deep brain stimulation device is further configured to adjust the electrical stimulation signal based on the stimulation parameter.

2. The deep brain stimulation algorithm system of claim 1, further comprising a server, wherein the server is coupled to the deep brain stimulation device and configured to store disease statistics and generate a personalization adjustment signal to the deep brain stimulation device based on the medical data and the disease statistics,
wherein the personalization module is further configured to adjust the prediction result based on the personalization adjustment signal.

3. The deep brain stimulation algorithm system of claim 2, wherein the deep brain stimulation device further comprises a wireless transmission module configured to provide a signal path between the deep brain stimulation device and the server, wherein the medical data and the personalization adjustment signal are transmitted between the deep brain stimulation device and the server through the signal path.

4. The deep brain stimulation algorithm system of claim 1, wherein the local field potential signal comprises an alpha brain wave signal, a beta brain wave signal and a gamma brain wave signal.

5. The deep brain stimulation algorithm system of claim 1, wherein the adjustment module is configured to generate the adjustment parameter to the control module in each adjustment cycle; or
wherein the adjustment module is configured to generate the adjustment parameter to the control module when receiving an adjustment command.

6. The deep brain stimulation algorithm system of claim 1, wherein the deep brain stimulation device further comprises an electrical stimulation module, wherein the electrical stimulation module is coupled between the control module and the subject, and is configured to generate the electrical stimulation signal to the subject based on the stimulation parameter.

7. An operation method of a deep brain stimulation algorithm system, comprising:
generating, by a deep brain stimulation device, an electrical stimulation signal to a subject;
sensing, by a signal sensing module of the deep brain stimulation device, a local field potential signal from the subject;
generating, by a control module of the deep brain stimulation device, a stimulation parameter;
generating, by a personalization module of the deep brain stimulation device, a prediction result based on a medical data and the local field potential signal;
comparing, by an adjustment module of the deep brain stimulation device, the local field potential signal and the prediction result by using a full spectrum analysis, so as to generate an adjustment parameter to the control module;
adjusting, by the control module, the stimulation parameter based on the adjustment parameter; and
adjusting, by the deep brain stimulation device, the electrical stimulation signal based on the stimulation parameter.

8. The operation method of claim 7, wherein after generating, by the personalization module of the deep brain stimulation device, the prediction result based on the medical data and the local field potential signal, the operation method further comprises:
generating, by a server, a personalization adjustment signal to the deep brain stimulation device based on the medical data and disease statistics, wherein the disease statistics is stored in the server; and
adjusting, by the personalization module, the prediction result based on the personalization adjustment signal.

9. The operation method of claim 8, wherein the medical data and the personalization adjustment signal are transmitted between the deep brain stimulation device and the server through a signal path provided by a wireless transmission module of the deep brain stimulation device.

10. The operation method of claim 7, wherein the local field potential signal comprises an alpha brain wave signal, a beta brain wave signal and a gamma brain wave signal.

11. The operation method of claim 7, wherein comparing, by the adjustment module of the deep brain stimulation device, the local field potential signal and the prediction result by using the full spectrum analysis, so as to generate the adjustment parameter to the control module is performed in response to each adjustment cycle; or
comparing, by the adjustment module of the deep brain stimulation device, the local field potential signal and the prediction result by using the full spectrum analysis, so as to generate the adjustment parameter to the control module is performed in response to the adjustment module receiving an adjustment command.

12. The operation method of claim 7, wherein generating, by the deep brain stimulation device, the electrical stimulation signal to the subject comprises:
generating, by an electrical stimulation module of the deep brain stimulation device, the electrical stimulation signal to the subject based on the stimulation parameter.

13. A non-transient computer readable storage medium, storing a plurality of computer readable instructions, when the plurality of computer readable instructions are executed for performing a deep brain stimulation algorithm on a subject by one or a plurality of processors, the one or the plurality of processors is configured to perform the following operations:
generating an electrical stimulation signal to the subject;
sensing a local field potential signal from the subject;
generating a stimulation parameter;
generating a prediction result based on a medical data and the local field potential signal;
comparing the local field potential signal and the prediction result by using a full spectrum analysis, so as to generate an adjustment parameter;
adjusting the stimulation parameter based on the adjustment parameter; and
adjusting the electrical stimulation signal based on the stimulation parameter.

14. The non-transient computer readable storage medium of claim 13, wherein after generating the prediction result based on the medical data and the local field potential signal, the one or the plurality of processors is further configured to perform the following operations:
generating a personalization adjustment signal based on the medical data and disease statistics; and
adjusting the prediction result based on the personalization adjustment signal.

15. The non-transient computer readable storage medium of claim 14, wherein the medical data and the personalization adjustment signal are transmitted through a signal path provided by a wireless transmission module.

16. The non-transient computer readable storage medium of claim 13, wherein the local field potential signal comprises an alpha brain wave signal, a beta brain wave signal and a gamma brain wave signal.

17. The non-transient computer readable storage medium of claim 13, wherein the adjustment parameter is configured to be generated in each adjustment cycle, or configured to be generated in response to an adjustment command.

18. The non-transient computer readable storage medium of claim 13, wherein generating the electrical stimulation signal to the subject comprises:
generating the electrical stimulation signal to the subject based on the stimulation parameter.
